(19) United States
(12) Patent Application Publication
(10) Pub. No.: US 2024/0197370 A1
(43) Pub. Date: Jun. 20, 2024

(54) OPERATION METHOD FOR BONE TISSUE AND OPERATION SYSTEM FOR BONE TISSUE

(71) Applicant: Shanghai DroidSurg Medical Co., Ltd., Shanghai (CN)

(72) Inventor: Shaobai WANG, Shanghai (CN)

(73) Assignee: Shanghai DroidSurg Medical Co., Ltd., Shanghai (CN)

(21) Appl. No.: 17/791,405

(22) PCT Filed: Jun. 2, 2021
(86) PCT No.: PCT/CN2021/097895

§ 371 Date: Jul. 7, 2022

(51) Int. Cl. A61B 17/70; A61B 17/00; A61B 17/16; A61B 17/86; A61B 34/00; A61B 34/37

(52) U.S. Cl. CPC A61B 17/7071; A61B 17/1671; A61B 17/8665; A61B 34/25; A61B 34/37; A61B 2017/00367; A61B 2034/252

(57) ABSTRACT

The present disclosure provides an operation method for bone tissue. The operation method for bone tissue includes: providing a first implantation device and a second implantation device, where the first implantation device includes a hollowed structure, an adapter structure is arranged in the hollowed structure, and at least a portion of an outer side wall of a bottom of the second implantation device is provided with a corresponding adapter structure to be fixedly connected to the adapter structure; proceeding to a location step, where the first implantation device penetrates a first vertebral segment, so as to cause the hollowed structure to face an end face of a second vertebral segment and make surface contact with the second vertebral segment, pre-locate the first vertebral segment and the second vertebral segment, and make the first vertebral segment and the second verteral segment self-adapted to adjacent vertebrae; and proceeding to a fixation step, where the second implantation device penetrates from the bottom thereof the hollowed structure to be fixedly connected to the second vertebral segment, and the second implantation device is fixedly connected to the hollowed structure by means of fixed connection between the corresponding adapter structure and the adapter structure, thereby reducing a stress and avoiding re-adjustment of adjacent vertebrae in location. The present disclosure further provides an operation system for bone tissue.

### TECHNICAL FIELD

The present disclosure relates to the medical field, in particular to an operation method and an operation system for bone tissue.

### BACKGROUND

In some orthopedic operations, medical instruments are required for re-combining two separated vertebral segments for fixation. For example, in the process of treating spinal stenosis, it is required to cut off a target spinal segment before operation treatment, and then fix the two cut spinal segments. In existing operation methods for bone tissue, screws are generally used to fix two spinal segments. Such methods cannot adjust offset of the two spinal segments caused during space formation, such that after direct fixation, there will be offset between the two spinal segments, postoperative risks are likely to occur, or it is necessary to adjust a location between adjacent vertebrae, thereby increasing the operation difficulty, prolonging operation time and improving input cost.

Disclosed in a patent application with Publication No. CN1455656A is a method for spacing a spinal canal, which re-fixes the separated vertebral space. In the above method, an implant including a stent, a washer, a screw and a rope is used, the stent is squeezed into the space, the screw is inserted into the washer and the stent and squeezed into the front of a vertebra for each space of the vertebra, and the rope is connected to the washer at each end and tied to penetrate the back of the vertebra to fix the back of the vertebra to its front, thereby re-stabilizing the cut spinal canal. However, the above method is complicated in operation, and is likely to form significant stress on the spinal canal, which affects postoperative experience of patients. Moreover, since the two spinal segments are fixed with the screws, postoperative risks are likely to occur due to offset between the two spinal segments, or it is necessary to adjust the location between adjacent vertebrae, thereby increasing the operation difficulty, prolonging operation time and improving input cost.

To this end, it is necessary to develop a novel operation method and an operation system for bone tissue, so as to solve the above problems in the prior art.

### SUMMARY

An objective of the present disclosure is to provide an operation method and an operation system for bone tissue, so to solve the problems that an operation method and an operation system for bone tissue in the prior art are complicated in operation, significant stress is likely to form on a spinal canal, postoperative experience of patients are affected consequently, in addition, postoperative risks are likely to occur due to offset between two spinal segments, or it is necessary to adjust a location between adjacent vertebrae.

To achieve the above objective, the operation method for bone tissue of the present disclosure includes:
S0: providing a first implantation device and a second implantation device, where the first implantation device includes a hollowed structure, an adapter structure is arranged in the hollowed structure, and at least a portion of an outer side wall of a bottom of the second implantation device is provided with a corresponding adapter structure to be fixedly connected to the adapter structure;
S1: proceeding to a location step, where the first implantation device penetrates a first vertebral segment, so as to cause the hollowed structure to face an end face of a second vertebral segment and make surface contact with the second vertebral segment; and
S2: proceeding to a fixation step, where the second implantation device penetrates from the bottom thereof the hollowed structure to be fixedly connected to the second vertebral segment, and the second implantation device is fixedly connected to the hollowed structure by means of fixed connection between the corresponding adapter structure and the adapter structure.

The operation method for bone tissue of the present disclosure has the beneficial effects: S1, the location step proceeds, where the first implantation device penetrates the first vertebral segment, so as to cause the hollowed structure to face the end face of the second vertebral segment and make surface contact with the second vertebral segment, such that damage to a post-osteotomy section may be avoided, the first vertebral segment and the second vertebral segment may be pre-located by means of the first implantation device, and the first vertebral segment and the second vertebral segment separated by the space may pre-adapt to adjacent vertebrae thereof correspondingly by implanting the first implantation device; and S2, the fixation step proceeds, where the second implantation device penetrates from the bottom thereof to the hollowed structure to be fixedly connected to the second vertebral segment, the second implantation device is fixedly connected to the hollowed structure by means of fixed connection between the corresponding adapter structure and the adapter structure, that is, while the first vertebral segment and the second vertebral segment separated by the space are relatively re-fixed by means of interaction between the second implantation device and the first implantation device, the stress is reduced, the postoperative experience of the patients are prevented from being influenced, and the second vertebral segment may adapt to adjacent vertebrae thereof, thereby directly adjusting the location between the second vertebral segment and the adjacent vertebrae, avoiding special re-adjustment of the location between the adjacent vertebrae, shortening operation time, eliminating demand for auxiliary adjustment instruments and reducing cost input.

Preferably, the operation method for bone tissue further includes a vertebral segment spacing step after the location step in S1 and before the fixation step in S2, where the vertebral segment spacing step includes: adjusting a moving distance of the first implantation device towards the second vertebral segment, causing the hollowed structure to move towards the second vertebral segment, and spacing the first vertebral segment and the second vertebral segment by a vertebral segment distance. The operation system for bone tissue has the following beneficial effect as follows: the vertebral segment distance between the first vertebral segment and the second vertebral segment is controlled by means of the vertebral segment spacing step.

Preferably, during movement of the hollowed structure towards the second vertebral segment, the hollowed structure is controlled to face the end face of the second vertebral segment and keep surface contact with the second vertebral segment. The operation system for bone tissue has the following beneficial effect as follows: damage to the post-osteotomy section is avoided.

Preferably, before the location step in S1 is performed, an osteotomy device is used to act on target bone tissue to form a space.

Preferably, in the location step in S1, the step of causing the first implantation device to penetrate a first vertebral segment includes: causing the first implantation device to move from a location, near a transverse process, of a lamina in a direction towards the space until the first implantation device penetrates the first vertebral segment. The operation method for bone tissue has the beneficial effects as follows: a target location is easy to find during an operation and more accurate location is achieved.

Preferably, in the location step in S1, the hollowed structure is caused to face the end face of the second vertebral segment and make planar contact or spherical contact with the second vertebral segment. The operation system for bone tissue has the following beneficial effect as follows: damage to the post-osteotomy section is avoided.

Preferably, before the location step in S1 is performed, the method further includes a preoperative path planning step, the preoperative path planning step including:
providing three-dimensional model data and vertebral segment distance requirement data of target bone tissue;
constructing section data according to the three-dimensional model data;
forming second vertebral segment section contact data according to the vertebral segment distance requirement data and the section data; and
forming first vertebral segment implantation path data according to the second vertebral segment section contact data.

Preferably, the present disclosure further provides the operation system for bone tissue. The operation system for bone tissue includes a first implantation device and a second implantation device, where the first implantation device includes a hollowed structure, an adapter structure is arranged in the hollowed structure, and at least a portion of an outer side wall of a bottom of the second implantation device is provided with a corresponding adapter structure to be fixedly connected to the adapter structure.

The operation system for bone tissue of the present disclosure has the beneficial effects: the operation system for bone tissue includes the first implantation device and the second implantation device, where the first implantation device includes the hollowed structure, and the adapter structure is arranged in the hollowed structure, such that the first implantation device may be used by a user in hand or by means of a robot, etc. to penetrate the first vertebral segment, and the hollowed structure is caused to face the end face of the second vertebral segment and make surface contact with the second vertebral segment to perform a location step, such that damage to a post-osteotomy section may be avoided, the first vertebral segment and the second vertebral segment may be pre-located by means of the first implantation device, and the first vertebral segment and the second vertebral segment separated by the space may pre-adapt to adjacent vertebrae thereof correspondingly by implanting the first implantation device; and at least the portion of the outer side wall of the bottom of the second implantation device is provided with the corresponding adapter structure to be fixedly connected to the adapter structure, such that the second implantation device may be used by the user in hand or by means of the operation robot, etc. to penetrate the hollowed structure to be fixedly connected to the second vertebral segment, the second implantation device is caused to be fixedly connected to the hollowed structure by means of fixed connection between the corresponding adapter structure and the adapter structure to perform a fixation step, that is, while the first vertebral segment and the second vertebral segment separated by the space are relatively re-fixed by means of interaction between the second implantation device and the first implantation device, the stress is reduced, postoperative experience of patients are prevented from being influenced, and the second vertebral segment may adapt to adjacent vertebrae thereof, thereby directly adjusting the location between the second vertebral segment and the adjacent vertebrae, avoiding special re-adjustment of the location between the adjacent vertebrae, shortening operation time, eliminating demand for auxiliary adjustment instruments and reducing cost input.

Preferably, the operation system for bone tissue further includes a master control unit and an implantation mechanical arm, the master control unit being in communication with the implantation mechanical arm; where the implantation mechanical arm is provided with a clamping structure to adapt to the first implantation device and the second implantation device; and the master control unit controls the implantation mechanical arm to clamp the first implantation device to penetrate a first vertebral segment and to be fixedly connected to same, so as to form a spacing distance between the first vertebral segment and a second vertebral segment, and further the master control unit controls the implantation mechanical arm to clamp the second implantation device to penetrate the first implantation device and to be fixedly connected to same and the second vertebral segment. The operation system for bone tissue has the beneficial effects as follows: while the vertebral segments separated by the space are relatively re-fixed, the stress is reduced, and the postoperative experience of the patients is prevented from being influenced.

Preferably, the operation system for bone tissue further includes an auxiliary image device in communication with the master control unit, where the master control unit stores spacing distance range reference data; the auxiliary image device acquires real-time location information of the first vertebral segment and the second vertebral segment and feeds same back to the master control unit; and the master control unit determines whether the spacing distance conforms to the spacing distance range reference data according to the real-time location information. The operation system for bone tissue has the beneficial effect as follows: accuracy of the spacing distance is guaranteed, and the stress is reduced.

Preferably, the first implantation device includes a nut structure provided with internal threads and external nut threads, a bottom of the nut structure includes a nut contact surface, the second implantation device includes a screw structure provided with external threads, and the external threads adapt to the internal threads; and the master control unit controls, by means of the implantation mechanical arm, the nut structure to penetrate the first vertebral segment, to pass through a space to make surface contact with the second vertebral segment to form the spacing distance and to be fixedly connected to the first vertebral segment, and further the master control unit controls, by means of the implantation mechanical arm, the screw structure to be placed into the second vertebral segment by means of the nut structure, and finally to be fixedly connected to the second vertebral segment by means of adaptation of the internal threads to the external threads. The operation system for bone tissue has the beneficial effect as follows: the spacing distance may be determined accurately.

Preferably, the clamping structure of the implantation mechanical arm includes a casing structure, a stretchable structure accommodated in the casing structure and a lock structure in movable contact with the stretchable structure, the casing structure being detachably and fixedly connected to the first implantation device, and the stretchable structure stretching out to push out the lock structure to abut against an inner wall of the first implantation device. The operation system for bone tissue has the beneficial effects as follows: the clamping structure of the implantation mechanical arm may be detachably and fixedly connected to the first implantation device, such that movement of the first implantation device may be conveniently controlled by means of the clamping structure of the implantation mechanical arm in a more accurate manner, which is conducive to movement stability of the first implantation device during implantation.

Preferably, a top of the first implantation device is provided with an accommodation structure, and at least a portion of the lock structure pushed out as the stretchable structure stretches out is accommodated in the accommodation structure. The operation system for bone tissue has the beneficial effects as follows: the accommodation structure is arranged to accommodate the lock structure, such that firmness of abutting connection between the lock structure and the first implantation device may be better guaranteed, the clamping structure of the implantation mechanical arm may be connected and fixed to the first implantation device more conveniently, and subsequent operations may be facilitated accordingly.

Preferably, the clamping structure of the implantation mechanical arm further includes an electromagnetic control portion electrically connected to the stretchable structure, and the electromagnetic control portion controls the lock structure to enter or exit the accommodation structure by controlling the stretchable structure to move towards or away from the first implantation device. The operation system for bone tissue has the beneficial effects as follows: accurate control is facilitated to a higher extent and higher operation flexibility is achieved.

Preferably, the operation system for bone tissue further includes an osteotomy device for acting on target bone tissue to form a space.

Preferably, the operation system for bone tissue further includes a master control unit and an osteotomy mechanical arm clamping the osteotomy device, where the master control unit is in communication connection with the osteotomy mechanical arm, and the osteotomy device includes an actuator clamped at a free end of the osteotomy mechanical arm, and the master control unit controls the osteotomy mechanical arm to cause the actuator to act on the target bone tissue to form the space. The operation system for bone tissue has the following beneficial effect as follows: accuracy of an osteotomy process is ensured.

Preferably, the osteotomy mechanical arm further includes a buffer mechanism partially sleeving the actuator, the buffer mechanism includes a closed cavity and a drive portion penetrating the closed cavity, the drive portion divides the closed cavity into a first cavity and a second cavity, the actuator is sleeved with the drive portion, the first cavity is close to an actuation end of the actuator, and the second cavity is far away from the actuation end of the actuator. The operation system for bone tissue has the following beneficial effect as follows: risks of nerve and blood vessel damage due to overshooting during osteotomy is reduced.

Preferably, the buffer mechanism further includes a pressure sensing portion arranged on the actuator to acquire change of a pressure by the actuator acting on the target bone tissue.

Preferably, the buffer mechanism further includes a duct portion which is arranged in the closed cavity and is in communication with the first cavity and the second cavity separately, and the duct portion is provided with an opening and closing control portion which is in communication connection with the pressure sensing portion; and when the pressure sensing portion detects that the pressure by the actuator acting on the target bone tissue is reduced to a minimum value within a first period as for the change of the pressure by the actuator acting on the target bone tissue, the opening and closing control portion closes communication of the duct portion with the first cavity and the second cavity, such that the drive portion produces a reverse thrust on the actuator in a direction opposite an operation direction to weaken the action of the actuator on the target bone tissue.

Preferably, the operation system for bone tissue further includes a master control unit and several mechanical arms, where the master control unit is in communication connection with the mechanical arms, and the several mechanical arms are in an identical structure or different structures. The operation system for bone tissue has beneficial effects as follows: the number of mechanical arms may be increased according to the requirements from implantation, osteotomy, monitoring, transmission, suture or other functional operation devices, and various use requirements may be met.

### BRIEF DESCRIPTION OF DRAWINGS

FIG. 1 is a flowchart of an operation method for bone tissue in an embodiment of the present disclosure;

FIG. 2 is a structural block diagram of a first operation system for bone tissue in an embodiment of the present disclosure;

FIG. 3 is a structural schematic diagram for fixing a broken vertebral pedicle with an implantation device in some embodiments of the present disclosure;

FIG. 4 is a structural block diagram of a second operation system for bone tissue in an embodiment of the present disclosure;

FIG. 5 is a structural block diagram of a third operation system for bone tissue in an embodiment of the present disclosure;

FIG. 6 is a structural schematic diagram showing a nut structure acting on a first vertebral segment in some embodiments of the present disclosure;

FIG. 7 is an enlarged structural schematic diagram of portion A shown in FIG. 6;

FIG. 8 is a schematic diagram of the nut structure shown in FIG. 6;

FIG. 9 is a structural schematic diagram of a bottom of the nut structure shown in FIG. 8;

FIG. 10 is a schematic diagram of an operation state in which an implantation mechanical arm clamps the nut structure in some embodiments of the present disclosure;

FIG. 11 is an enlarged structural schematic diagram of portion B shown in FIG. 10;

FIG. 12 is a structural schematic diagram showing a screw structure acting on the nut structure and a second vertebral segment in some embodiments of the present disclosure;

FIG. 13 is a schematic diagram of the screw structure shown in FIG. 12;

FIG. 14 is a structural block diagram of a fourth operation system for bone tissue in an embodiment of the present disclosure;

FIG. 15 is a structural schematic diagram of an osteotomy device in an embodiment of the present disclosure;

FIG. 16 is a sectional view of a portion of the structure shown in FIG. 15;

FIG. 17 is a partial structural schematic diagram of a buffer drive portion shown in FIG. 15; and

FIG. 18 is a structural schematic diagram of another osteotomy device in an embodiment of the present disclosure.

### DETAILED DESCRIPTION OF THE EMBODIMENTS

To make objectives, technical solutions, and advantages of embodiments of the present disclosure clearer, the technical solutions in the embodiments of the present disclosure will be clearly and completely described. Apparently, the described embodiments are some rather than all of the embodiments of the present disclosure. Based on the embodiments of the present disclosure, all the other embodiments obtained by those of ordinary skill in the art without inventive effort are within the scope of the present disclosure. Unless otherwise defined, technical terms or scientific terms used herein should have ordinary meanings understood by those with ordinary skills in the field to which the present disclosure belongs. As used herein, similar words such as “include” mean that elements or objects appearing before the word cover elements or objects listed after the word and their equivalents, but do not exclude other elements or objects.

The embodiments of the present disclosure provide an operation method and an operation system for bone tissue, so to solve the problems that an operation method and an operation system for bone tissue in the prior art are complicated in operation, significant stress is likely to form on a spinal canal, postoperative experience of patients are affected consequently, in addition, postoperative risks are likely to occur due to offset between two spinal segments, or it is necessary to adjust a location between adjacent vertebrae.

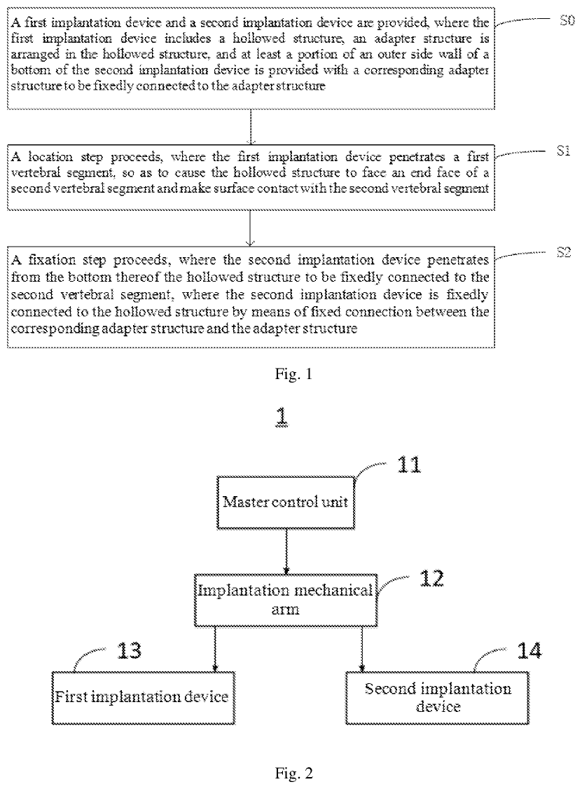

FIG. 1 is a flowchart of the operation method for bone tissue in the embodiment of the present disclosure.

With reference to FIG. 1, the operation method for bone tissue in the embodiment of the present disclosure includes:
S0: a first implantation device and a second implantation device are provided, where the first implantation device includes a hollowed structure, an adapter structure is arranged in the hollowed structure, and at least a portion of an outer side wall of a bottom of the second implantation device is provided with a corresponding adapter structure to be fixedly connected to the adapter structure;
S1: a location step proceeds, where the first implantation device penetrates a first vertebral segment, so as to cause the hollowed structure to face an end face of a second vertebral segment and make surface contact with the second vertebral segment; and
S2: a fixation step proceeds, where the second implantation device penetrates from the bottom thereof the hollowed structure to be fixedly connected to the second vertebral segment, the second implantation device is fixedly connected to the hollowed structure by means of fixed connection between the corresponding adapter structure and the adapter structure.

In some embodiments of the present disclosure, before the location step in S1 is performed, an osteotomy device is used to act on target bone tissue to form a space, the operation method for bone tissue acts on vertebrae with a space, and the space penetrates at least one vertebral pedicle, and one vertebral segment, including a lamina, of the two vertebral segments separated by the space is the first vertebral segment and a vertebral segment, including a vertebral body, of the two vertebral segments separated by the space is the second vertebral segment.

In some embodiments of the present disclosure, the operation method for bone tissue further includes a vertebral segment spacing step after the location step in S1 and before the fixation step in S2, where the vertebral segment spacing step includes: a moving distance of the first implantation device towards the second vertebral segment is adjusted, the hollowed structure is caused to move towards the second vertebral segment, and a vertebral segment distance between the first vertebral segment and the second vertebral segment is spaced. The vertebral segment distance between the first vertebral segment and the second vertebral segment is controlled in the vertebral segment spacing step, and the vertebral segment distance is planned by a doctor according to an image result of a patient with a goal of spacing by a vertebral foramen space and reducing compression.

In some embodiments of the present disclosure, in the location step in S1, the step of causing the first implantation device to penetrate a first vertebral segment includes: the first implantation device is used to move from a location, near a transverse process, of the lamina in a direction towards the space until the first implantation device penetrates the first vertebral segment.

In some embodiments of the present disclosure, the present disclosure further provides the operation system for bone tissue. The operation system for bone tissue includes a first implantation device and a second implantation device, where the first implantation device includes a hollowed structure, an adapter structure is arranged in the hollowed structure, and at least a portion of an outer side wall of a bottom of the second implantation device is provided with a corresponding adapter structure to be fixedly connected to the adapter structure.

In some embodiments of the present disclosure, a user holds the first implantation device to penetrate the first vertebral segment, and the hollowed structure is caused to face an end face of the second vertebral segment and make surface contact with the second vertebral segment to perform a location step. The user further holds the second implantation device to penetrate the hollowed structure and then to be fixedly connected to the second vertebral segment, and the second implantation device is caused to be fixedly connected to the hollowed structure by means of fixed connection between the corresponding adapter structure and the adapter structure to perform a fixation step.

In some other embodiments of the present disclosure, the operation system for bone tissue further includes an operation robot, the operation robot is used for causing the first implantation device to penetrate the first vertebral segment, and causing the hollowed structure to face the end face of the second vertebral segment and make surface contact with the second vertebral segment to perform the location step. The operation robot is further used for causing the second implantation device to penetrate the hollowed structure, and then be fixed connected to the second implantation device, and causing the second implantation device to be fixedly connected to the hollowed structure by means of fixed connection between the corresponding adapter structure and the adapter structure to perform the fixation step.

In some embodiments of the present disclosure, an operation system for bone tissue is provided for performing the operation method for bone tissue. The operation system for bone tissue includes a first implantation device, a second implantation device and an operation robot. The first implantation device includes a hollowed structure, an adapter structure is arranged in the hollowed structure, and at least a portion of an outer side wall of a bottom of the second implantation device is provided with a corresponding adapter structure to be fixedly connected to the adapter structure. The operation robot is used for causing the first implantation device to penetrate the first vertebral segment, and causing the hollowed structure to face an end face of the second vertebral segment and make surface contact with the second vertebral segment to perform a location step. The operation robot is further used for causing the second implantation device to penetrate the hollowed structure, and then be fixed connected to the second implantation device, and causing the second implantation device to be fixedly connected to the hollowed structure by means of fixed connection between the corresponding adapter structure and the adapter structure to perform a fixation step.

In some other embodiments of the present disclosure, the operation system for bone tissue further includes a master control unit and an implantation mechanical arm, the master control unit being in communication with the implantation mechanical arm; where the implantation mechanical arm is provided with a clamping structure to adapt to the first implantation device and the second implantation device; and the master control unit controls the implantation mechanical arm to clamp the first implantation device to penetrate a first vertebral segment and to be fixedly connected to same, so as to form a spacing distance between the first vertebral segment and a second vertebral segment, and further the master control unit controls the implantation mechanical arm to clamp the second implantation device to penetrate the first implantation device and to be fixedly connected to same and the second vertebral segment.

In some other embodiments of the present disclosure, the operation robot includes a master control unit and an implantation mechanical arm, and the implantation mechanical arm, the first implantation device and the second implantation device are all in communication connection with the master control unit. The implantation mechanical arm is provided with a clamping structure to adapt to the first implantation device and the second implantation device; and the master control unit controls the implantation mechanical arm to clamp the first implantation device to penetrate a first vertebral segment and to be fixedly connected to same, so as to form a spacing distance between the first vertebral segment and a second vertebral segment, and further the master control unit controls the implantation mechanical arm to clamp the second implantation device to penetrate the first implantation device and to be fixedly connected to same and the second vertebral segment.

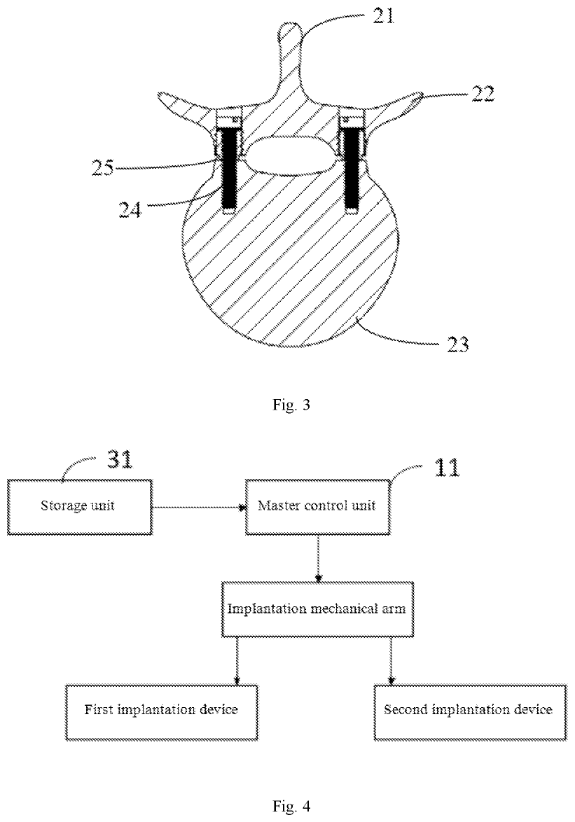

FIG. 2 is a structural block diagram of a first operation system for bone tissue in an embodiment of the present disclosure, and FIG. 3 is a structural schematic diagram for fixing a broken vertebral pedicle with an implantation device in some embodiments of the present disclosure.

With reference to FIGS. 2 and 3, the operation system for bone tissue 1 shown in FIG. 2 includes a master control unit 11 and an implantation mechanical arm 12, a first implantation device 13 and a second implantation device 14 which are communication connection with the master control unit 11. The first implantation device 13 and the second implantation device 14 constitute an implantation device 24. A first vertebral segment 22 and a second vertebral segment 23 are separated by a space 25.

The master control unit 11 controls the implantation mechanical arm 12 to clamp the first implantation device 13 to penetrate the first vertebral segment 22 and to be fixedly connected to same, so as to form a spacing distance between the first vertebral segment 22 and the second vertebral segment 23 and guarantee accuracy of the spacing distance, and further the master control unit controls the implantation mechanical arm 12 to clamp the second implantation device 14 to penetrate the first implantation device 13 and to be fixedly connected to same and the second vertebral segment 23.

In some embodiments of the present disclosure, the implantation mechanical arm is provided with a clamping structure to adapt to the first implantation device and the second implantation device.

In some embodiments of the present disclosure, before the location step is performed, the method further includes a preoperative path planning step. The preoperative path planning step includes:
three-dimensional model data and vertebral segment distance requirement data of target bone tissue are provided;
section data are constructed according to the three-dimensional model data;
second vertebral segment section contact data are formed according to the vertebral segment distance requirement data and the section data; and
first vertebral segment implantation path data are formed according to the second vertebral segment section contact data.

FIG. 4 is a structural block diagram of a second operation system for bone tissue in an embodiment of the present disclosure.

With reference to FIGS. 2 and 4, the operation system for bone tissue shown in FIG. 4 differs from the operation system for bone tissue 1 shown in FIG. 2 in that the operation system for bone tissue shown in FIG. 4 further includes a storage portion 31 in communication connection with the master control unit 11, and the storage portion 31 stores spacing distance range reference data. The master control unit 11 calls the spacing distance range reference data from the storage unit 31 to control the spacing distance to conform to the spacing distance range reference data, so as to guarantee accuracy of the actual spacing distance.

In some embodiments of the present disclosure, the master control unit 11 stores the spacing distance range reference data, that is, the storage unit 31 is a constituent structure in the master control unit 11.

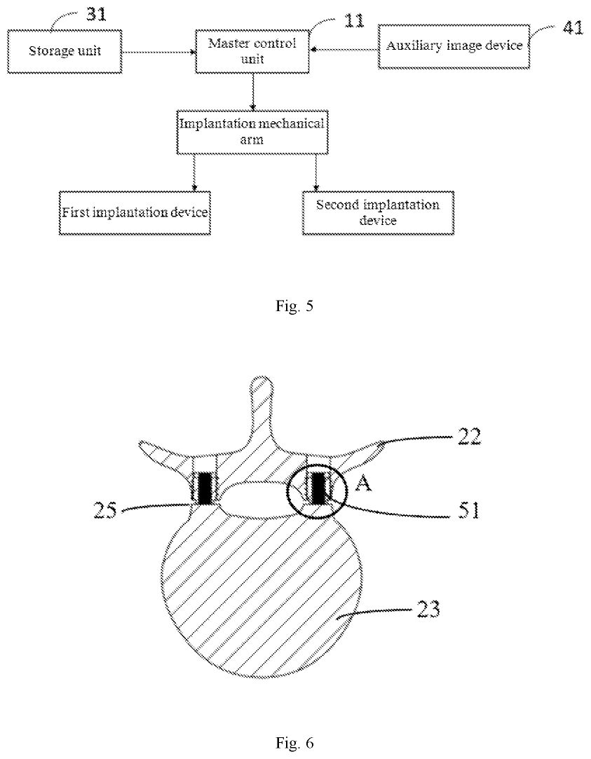

FIG. 5 is a structural block diagram of a third operation system for bone tissue in an embodiment of the present disclosure.

With reference to FIGS. 4 and 5, the operation system for bone tissue shown in FIG. 5 differs from that shown in FIG. 4 in that the operation system for bone tissue shown in FIG. 5 further includes an auxiliary image device 41 in communication connection with the master control unit 11.

With reference to FIGS. 3 and 5, the auxiliary image device 41 acquires real-time location information of the first vertebral segment 22 and the second vertebral segment 23 and feeds same back to the master control unit 11. The master control unit 11 determines whether the spacing distance conforms to the spacing distance range reference data according to the real-time location information, thus guaranteeing the accuracy of the spacing distance.

In some embodiments of the present disclosure, the auxiliary image device 41 is a C-arm X-ray machine or a computed tomography (CT) apparatus to acquire image information of target bone tissue.

In some embodiments of the present disclosure, during movement of the hollowed structure towards the second vertebral segment, the hollowed structure is controlled to face the end face of the second vertebral segment and keep surface contact with the second vertebral segment, thereby avoiding damage to a post-osteotomy section.

In some embodiments of the present disclosure, in the location step in S1, the hollowed structure is caused to face the end face of the second vertebral segment and make planar contact or spherical contact with the second vertebral segment, thereby avoiding damage to the post-osteotomy section.

In some embodiments of the present disclosure, the first implantation device includes a nut structure provided with internal threads and external nut threads, the second implantation device includes a screw structure provided with external threads, and the external threads adapt to the internal threads.

In some embodiments of the present disclosure, a bottom of the nut structure includes a nut contact surface, such that in the location step in S1, the hollowed structure is caused to face the end face of the second vertebral segment and make planar contact or spherical contact with the second vertebral segment, thereby avoiding damage to the post-osteotomy section.

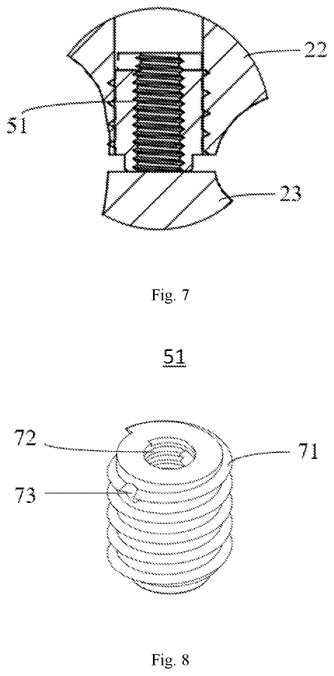

FIG. 6 is a structural schematic diagram showing a nut structure acting on a first vertebral segment in some embodiments of the present disclosure, and FIG. 7 is an enlarged structural schematic diagram of portion A shown in FIG. 6.

With reference to FIGS. 2, 6 and 7, the master control unit 11 controls, by means of the implantation mechanical arm 12, the nut structure 51 to penetrate the first vertebral segment 22, and pass through space 25 to make surface contact with the second vertebral segment 23 to form the spacing distance and be fixedly connected to the first vertebral segment 22. The bottom of the nut structure 51 makes surface contact with a top surface of the second vertebral segment 23. By controlling a depth of the nut structure 51 into the first vertebral segment 22, the spacing distance between the first vertebral segment 22 and the second vertebral segment 23, that is, the vertebral segment distance between the first vertebral segment 22 and the second vertebral segment 23, is controlled.

In some embodiments of the present disclosure, the spacing distance between the first vertebral segment 22 and the second vertebral segment 23 is an average of several vertical distances from a bottom of the first vertebral segment 22 to the top surface of the second vertebral segment 23.

In some embodiments of the present disclosure, the clamping structure of the implantation mechanical arm includes a casing structure, a stretchable structure accommodated in the casing structure and a lock structure in movable contact with the stretchable structure, the casing structure being detachably and fixedly connected to the first implantation device, and the stretchable structure stretching out to push out the lock structure to abut against an inner wall of the first implantation device, such that the clamping structure of the implantation mechanical arm may be detachably and fixedly connected to the first implantation device, movement of the first implantation device may be conveniently controlled by means of the clamping structure of the implantation mechanical arm in a more accurate manner, which is conducive to movement stability of the first implantation device during implantation.

In some embodiments of the present disclosure, a top of the first implantation device is provided with an accommodation structure, at least a portion of the lock structure pushed out as the stretchable structure stretches out is accommodated in the accommodation structure, the accommodation structure is arranged to accommodate the lock structure, such that firmness of abutting connection between the lock structure and the first implantation device may be better guaranteed, the clamping structure of the implantation mechanical arm may be connected and fixed to the first implantation device more conveniently, and subsequent operations may be facilitated accordingly.

In some embodiments of the present disclosure, the clamping structure of the implantation mechanical arm further includes an electromagnetic control portion electrically connected to the stretchable structure, and the electromagnetic control portion controls the lock structure to enter or exit the accommodation structure by controlling the stretchable structure to move towards or away from the first implantation device, such that accurate control is facilitated to a higher extent and higher operation flexibility is achieved.

In some embodiment of the present disclosure, the lock structure includes several ball structure relatively attracted to two sides of the stretchable structure. When the electromagnetic control portion controls the stretchable structure to move towards the first implantation device, a portion of the stretchable structure is accommodated in the top of the first implantation device, and the several ball structures enter the accommodation structure from the two sides of the stretchable structure and contact with the stretchable structure to compress the stretchable structure.

In some embodiments of the present disclosure, the accommodation structure is arranged in a radial direction of the first implantation device, and an axial length inner diameter of the accommodation structure is smaller than a maximum axial length of the lock structure, such that firmness of abutting connection between the lock structure and the first implantation device may be better guaranteed, the clamping structure of the implantation mechanical arm may be connected and fixed to the first implantation device more conveniently, and subsequent operations may be facilitated accordingly.

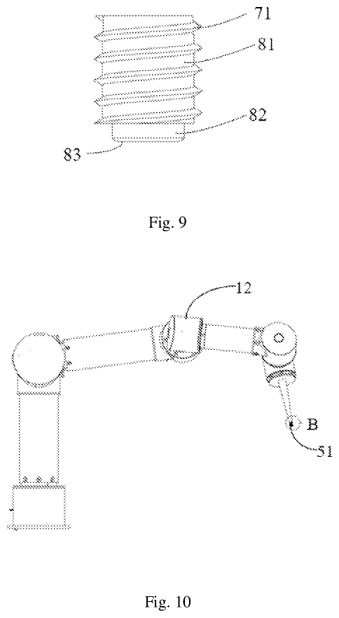

FIG. 8 is a schematic diagram of the nut structure shown in FIG. 6 and FIG. 9 is a structural schematic diagram of a bottom of the nut structure shown in FIG. 8.

With reference to FIGS. 7-9, an outer side wall of a nut body 81 of the nut structure 51 is provided with nut external threads 71, which is conducive to fixed connection between the nut structure 51 and the first vertebral segment 22 after the expansion distance is formed.

An inner side wall of the nut structure 51 is provided with internal threads 72 to adapt to the second implantation device.

A top of the nut structure 51 is provided with an accommodation structure 73 to adapt to the implantation mechanical arm 12.

A free end of the nut structure 51 converges towards an axis (not shown in the figure) of the nut structure to cause a portion of an outer side wall of the nut structure 51 to be arc-shaped, thereby reducing a friction effect of the surface contact between the nut structure 51 and the second vertebral segment 23.

Specifically, the bottom of the nut body 81 includes a boss 82, an outer diameter of the boss 82 is smaller than an outer diameter of the nut body 81, and a free end edge of the boss 82 is provided with a rounded structure 83, so as to cause the portion of the outer side wall of the nut structure 51 to be arc-shaped.

In some embodiments of the present disclosure, a height of the boss 82 is the expansion distance.

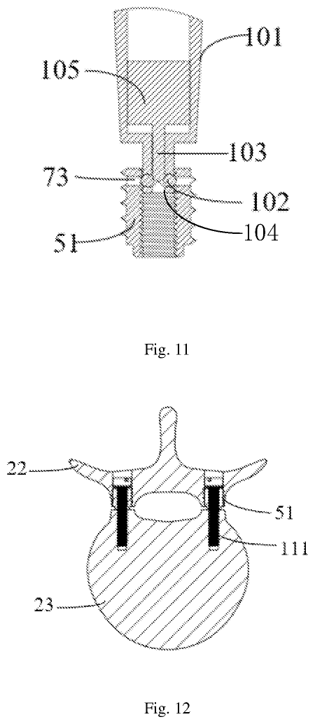

FIG. 10 is a schematic diagram of an operation state in which an implantation mechanical arm clamps the nut structure in some embodiments of the present disclosure. FIG. 11 is an enlarged structural schematic diagram of portion B shown in FIG. 10.

With reference to FIGS. 10 and 11, the implantation mechanical arm 12 is a six-axis mechanical arm. The clamping structure of the implantation mechanical arm 12 includes the casing structure 101, the stretchable structure 103 accommodated in the casing structure, and a lock structure 102 in movable contact with the stretchable structure 103. The casing structure 101 is in clearance fit with a cavity of the nut structure 51, that is, the casing structure 101 may be movably inserted into the cavity of the nut structure 51. The casing structure is further internally provided with a channel 104 allowing the stretchable structure 103 to stretch out and draw back, and the top of the nut structure 51 is provided with the accommodation structure 73. The stretchable structure 103 is electrically connected to the electromagnetic control portion 105.

Further, with reference to FIG. 11, the accommodation structure 73 is arranged in a radial direction of the nut structure 51. Specifically, an inner diameter of the accommodation structure 73 is smaller than a diameter of the ball structure 102.

In some embodiments of the present disclosure, the stretchable structure 103 and the electromagnetic control portion 105 constitute an electromagnetic bolt.

Specifically, the electromagnetic control portion 105 causes the stretchable structure 103 to be accommodated in the casing structure 101 when not energized, and the two ball structures 102 are attracted to the bottom of the casing structure 101.

When the nut structure 51 needs to be clamped, the bottom of the casing structure 101 is accommodated in the top of the nut structure 51, and after the electromagnetic control portion 105 is energized, the stretchable structure 103 stretches out of the bottom of the casing structure 101 and moves towards the bottom of the nut structure 51 under the action of electromagnetic attraction. Since the inner diameter of the accommodation channel 73 is smaller than the diameter of the ball structure 102, after one portions of the two ball structures 102 are squeezed into the accommodation structure 73, the other portions clamp the stretchable structure 103 on the opposite sides of the stretchable structure 103, thus clamping and fixing the nut structure 51.

In some embodiments of the present disclosure, the accommodation structure penetrates the top of the nut structure, such that a penetrated structure is formed, and a process is simple and machining is more convenient.

In some embodiments of the present disclosure, the second implantation device 14 includes the screw structure.

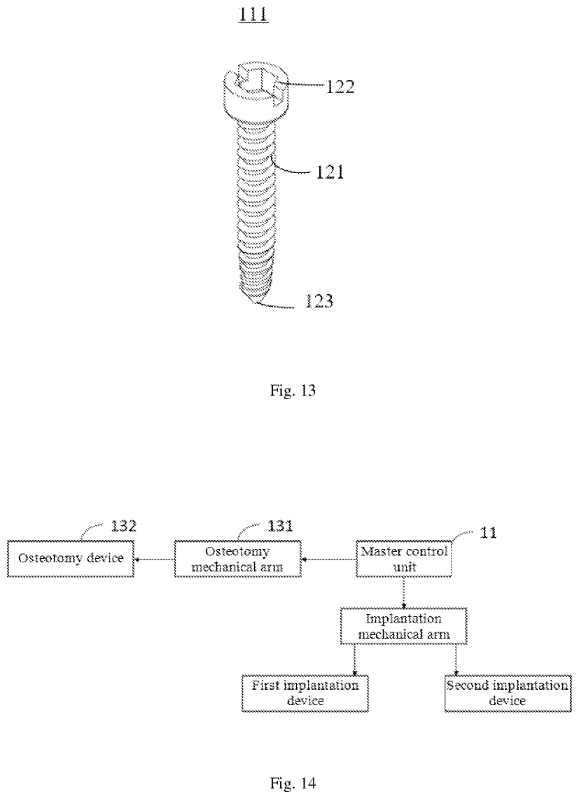

FIG. 12 is a structural schematic diagram for showing a screw structure acting on the nut structure and a second vertebral segment in some embodiments of the present disclosure, and FIG. 13 is a schematic diagram of the screw structure shown in FIG. 12

With reference to FIGS. 2, 12 and 13, after the nut structure 51 is implanted into the first vertebral segment 22, the implantation mechanical arm 12 controls the screw structure 111 to be implanted into the second vertebral segment 23 by means of the nut structure 51. The screw structure 111 is provided with the external threads 121 to adapt to the internal threads (not shown in the figure) of the nut structure 51. The screw structure 111 has a bottom in a pointed structure 123, so as to be smoothly screwed into the second vertebral segment 23. By means of the external threads 121, the screw structure 111 may be fixedly connected to the second vertebral segment 23 and the nut structure 51, such that the first vertebral segment 22 and the second vertebral segment 23 may be fixed.

In some embodiments of the present disclosure, with reference to FIG. 13, a top of the screw structure 111 is provided with a screw accommodation structure 122 with the same structure as that of the accommodation structure, so as to accommodate the lock structure and to be detachably and fixedly connected to the casing structure, such that the clamping structure of the implantation mechanical arm may be detachably and fixedly connected to the screw structure, and movement of the screw structure may be conveniently controlled by means of the clamping structure of the implantation mechanical arm in a more accurate manner, which is conducive to movement stability of the screw structure during implantation.

In some embodiments of the present disclosure, before the location step in S1 is performed, an ultrasonic osteotome is used to act on the target bone tissue to form the space.

With reference to FIG. 3, after the ultrasonic osteotome is used to form the space 25 shown in FIG. 3 during bone tissue operation, the vertebral pedicle 21 forms the first vertebral segment 22 and the second vertebral segment 23, and then the implantation device 24 penetrates the first vertebral segment 22, and passes through the space 25 to be fixed to the second vertebral segment 23.

However, since there are many nerves and blood vessels around the vertebral pedicle 21, these nerves and blood vessels are needed to be protected from damage during the operation. During osteotomy with the osteotome, at the moment when the vertebral pedicle 21 is cut off, since thrust of the osteotome on bone suddenly disappears, the osteotome may overshoot and continue to move in the direction of action, such that damage to the nerves and the blood vessels and unnecessary damage to patients are likely to occur.

In some embodiments of the present disclosure, the operation system for bone tissue further includes an osteotomy device for acting on target bone tissue to form a space.

In some embodiments of the present disclosure, the operation system for bone tissue further includes the master control unit and the osteotomy mechanical arm clamping the osteotomy device, where the master control unit is in communication connection with the osteotomy mechanical arm, and the osteotomy device includes an actuator clamped at a free end of the osteotomy mechanical arm, and the master control unit controls the osteotomy mechanical arm to cause the actuator to act on the target bone tissue to form the space.

FIG. 14 is a structural block diagram of a fourth operation system for bone tissue in an embodiment of the present disclosure.

In some embodiments of the present disclosure, with reference to FIGS. 2, 3 and 14, the operation system for bone tissue shown in FIG. 14 differs from the operation system for bone tissue 1 shown in FIG. 2 is that the operation system for bone tissue shown in FIG. 14 further includes an osteotomy mechanical arm 131 which is in communication connection with a master control unit 11 and holds an osteotomy device 132, and the master control unit 11 controls the osteotomy mechanical arm 131 to cause the osteotomy device 132 to form a space 25 at a vertebral pedicle 21.

In some embodiments of the present disclosure, the osteotomy device 132 includes an actuator to clamp a free end of the osteotomy mechanical arm 131 and act on target bone tissue.

In some embodiment of the present disclosure, the actuator includes an ultrasonic osteotome.

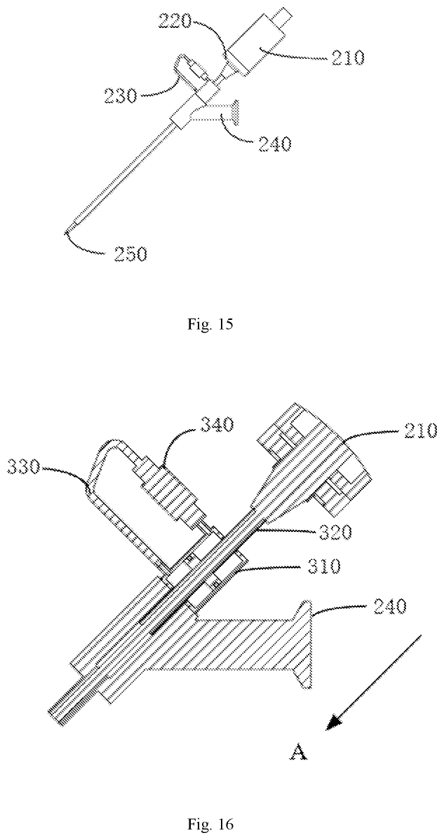

FIG. 15 is a structural schematic diagram of an osteotomy device in an embodiment of the present disclosure. FIG. 16 is a sectional view of a portion of the structure shown in FIG. 15.

With reference to FIGS. 15 and 16, the osteotomy device shown in FIG. 15 includes an actuator 210 and a buffer mechanism (not shown in the figure), and the buffer mechanism (not shown in the figure) partially sleeves the actuator 210. The buffer structure (not shown in the figure) is composed of a pressure sensing portion 220 and a buffer drive portion 230, and the buffer drive portion 230 is fixed to the actuator 210 in a sleeving manner. The pressure sensing portion 220 is arranged on the actuator 210.

In some embodiments of the present disclosure, the buffer mechanism acts on the actuator 210 according to detected change of the pressure by the actuator 210 acting on the target bone tissue.

In some embodiments of the present disclosure, with reference to FIGS. 15 and 16, the actuator 210 acts on the target bone tissue (not shown in the figure) in the operation direction, that is, an A direction shown in FIG. 16, and the pressure sensing portion 220 detects that the change of the pressure by the actuator 210 acting on the target bone tissue is a stable pressure condition, and feeds the stable pressure condition back to the buffer drive portion 230. The buffer drive portion 230 assists the actuator 210 in strengthening the action on the target bone tissue (not shown in the figure), that is, the buffer mechanism drives the actuator 210 to move in the A direction to act on the target bone tissue (not shown in the figure).

Specifically, the stable pressure condition means that a pressure value remains unchanged within a certain period of time or within a certain pressure control range, which indicates that the actuator 210 continuously and stably acts on the target bone tissue. Since the buffer drive portion 230 is fixed to the actuator 210 in a sleeving manner, the buffer drive portion may follow movement of the actuator 210 to stabilize the action on the target bone tissue (not shown in the figure).

In some embodiments of the present disclosure, with reference to FIGS. 15 and 16, the actuator 210 acts on the target bone tissue (not shown in the figure) in the A direction shown in FIG. 16, and the pressure sensing portion 220 detects that the change of the pressure by the actuator 210 acting on the target bone tissue is a sudden pressure fall condition, and feeds the sudden pressure fall condition back to the buffer drive portion 230. The buffer drive portion 230 produces reverse thrust in a direction opposite the A direction to weaken the action of the actuator 210 on the target bone tissue (not shown in the figure), thereby reducing the risk of nerve and blood vessel damage due to overshoot during osteotomy.

Specifically, the sudden pressure fall is that the pressure produced by the actuator 210 acting on the target bone tissue is reduced to a minimum value in a first period, that is, that is, the pressure acting on the target bone tissue at the moment when the actuator 210 cuts off the target bone tissue may be attenuated to zero or close to zero, and at this time, it is necessary to reduce the risk of nerve and blood vessel damage by means of the reverse thrust of the buffer drive portion 230 on the actuator 210.

In some embodiments, the minimum value is 0, and the first period is no longer than 0.1 s.

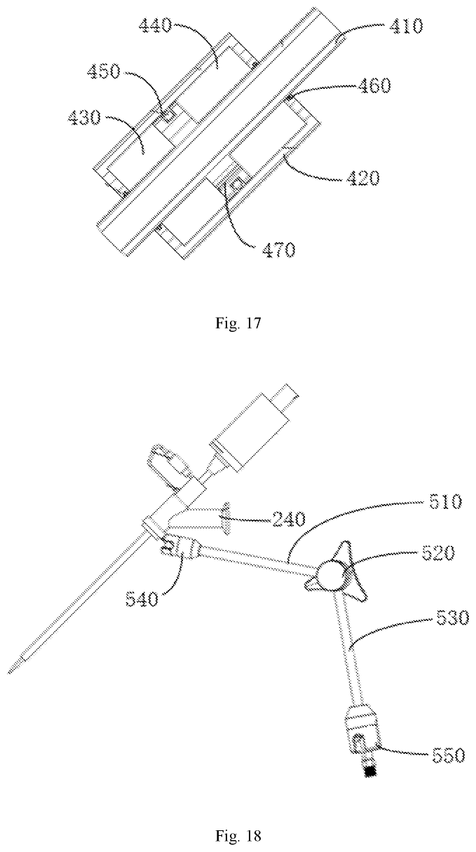

FIG. 17 is a partial structural schematic diagram of a buffer drive portion shown in FIG. 15.

With reference to FIGS. 15, 16 and 17, the buffer drive portion 230 includes a closed cavity 420 and a drive portion 410 penetrating the closed cavity 420, the drive portion 410 divides the closed cavity 420 into a first cavity 430 and a second cavity 440, the first cavity 430 is close to an actuation end 250 of the actuator 210, and the second cavity 440 is far away from the actuation end 250 of the actuator 210.

The buffer drive portion 230 further includes a duct portion 330 and an opening and closing control portion (not shown in the figure) which is arranged in the duct portion 330 and in communication connection with the pressure sensing portion 220, the duct portion 330 is arranged in the closed cavity 420 and is in communication connection with the first cavity 430 and the second cavity 440 separately, the opening and closing control portion (not shown in the figure) is composed of a control portion (not shown in the figure) and an opening and closing portion 340 which are in communication connection, and the control portion (not shown in the figure) is in communication connection with the pressure sensing portion 220.

In some specific embodiments, the control portion (not shown in the figure) is a computer control system.

In some specific embodiments, the opening and closing portion 340 is an electromagnetic valve.

In some specific embodiments, the pressure sensing portion 220 is a force sensor.

When the pressure sensing portion 220 detects that the pressure by the actuator 210 acting on the target bone tissue is reduced to the minimum value within the first period as for the change of the pressure by the actuator 210 acting on the target bone tissue, the opening and closing control portion, specifically the control portion, controls the opening and closing portion 340 to close communication of the duct portion 330 with the first cavity 430 and the second cavity 440, such that the drive portion 410 produces the reverse thrust on the actuator 210 in the direction opposite the operation direction to weaken the action of the actuator 210 on the target bone tissue.

In some embodiments, both the first cavity 430 and the second cavity 440 contain hydraulic media. When the opening and closing portion 340 opens the communication of the duct portion 330 with the first cavity 430 and the second cavity 440, the hydraulic media is transferred between the first cavity 430 and the second cavity 440 by means of the duct portion 330.

In some specific embodiments, the hydraulic medium is hydraulic oil.

In some specific embodiments, when the pressure sensing portion 220 detects that the change of the pressure by the actuator 210 acting on the target bone tissue is the stable pressure condition, the buffer drive portion 230 moves along with the actuator 210 in the operation direction to expand a volume in the second cavity 440 and compress a volume in the first cavity 430, in addition, since the volume in the first cavity 430 is compressed, the hydraulic medium therein flows into the second cavity 440 by means of the duct portion 330 to further compress the volume in the first cavity 430, thereby assisting the actuator 210 in moving further in the operation direction to act on the target bone tissue.

In some specific embodiments, when the pressure sensing portion 220 detects and feeds back, to the control portion, that the pressure by the actuator 210 acting on the target bone tissue is reduced to the minimum value within the first period as for the change of the pressure by the actuator 210 acting on the target bone tissue, and the control portion controls the opening and closing portion 340 to close the communication of the duct portion 330 with the first cavity 430 and the second cavity 440, such that the hydraulic medium may not transfer between the first cavity 430 and the second cavity 440.

Just before the pressure produced by the actuator 210 acting on the target bone tissue is reduced to the minimum value within the first period, the hydraulic medium flows into the second cavity 440 by means of the duct portion 330 to further compress the volume in the first cavity 430. At the moment when the hydraulic medium between the first cavity 430 and the second cavity 440 may not be transferred to each other, a pressure in the first cavity 430 is greater than that in the second cavity 440, so as to produce the reverse thrust in the direction opposite the operation direction on the drive portion 410, then push the actuator 210 to move in the direction opposite the operation direction or stop the actuator 210 from moving, and weaken the action of the actuator 210 on the target bone tissue.

In some embodiments, with reference to FIG. 17, the drive portion 410 includes a division portion 470, and an outer side wall of the division portion 470 is attached to an inner side wall of the closed cavity 420, such that division portion moves relative to the closed cavity 420 and divides the closed cavity 420 to form the first cavity 430 and the second cavity 440.

In some embodiments, the drive portion 410 further includes a hollowed shaft part to penetrate and be fixedly connected to the division portion 470, and penetrate and be movably connected to the closed cavity 420.

In some embodiments of the present disclosure, seal structures are arranged at a joint and an attachment portion between the drive portion 410 and the closed cavity 420.

Specifically, with reference to FIG. 16 and FIG. 17, a first seal structure 460 is arranged at the joint of the drive portion 410 and the closed cavity 420 to enhance a sealing effect on the closed cavity 420. The outer side wall of the division portion 470 is provided with a second seal structure 450 to attach to the closed cavity 420 and further enhance respective sealing of the first cavity 430 and the second cavity 440.

In some embodiments of the present disclosure, the first seal structure 460 and the second seal structure 450 are both seal rings.

In some embodiments of the present disclosure, the osteotomy device shown in FIG. 15 further includes an observation portion 240 to monitor the action of the actuator 210 on the target bone tissue (not shown in the figure).

With reference to FIGS. 15, 16 and 17, the observation portion 240 is fixedly connected to the drive portion 410, and the actuator 210 moves through the observation portion 240.

In some embodiments of the present disclosure, the observation portion 240 is an arthroscope with a hollowed structure.

In some embodiments of the present disclosure, the osteotomy device further includes an ultrasonic generator in communication connection with the actuator, such that an actuation end 250 of the actuator 210 acts on the target bone tissue in a high-frequency resonance mode. Specifically, the ultrasonic generator and the actuator 210 together constitute the ultrasonic osteotome.

In some embodiments of the present disclosure, the osteotomy device further includes a movable support portion movably connected to the observation portion 240 to adjust an orientation of the observation portion.

Specifically, a degree of freedom of motion of the movable support portion is 7, such that the orientation of the observation portion may be adjusted at will.

Specifically, the movable support portion includes a rotary joint portion and a plurality of ball joint portions.

In some embodiments of the present disclosure, the operation system for bone tissue further includes a master control unit and several mechanical arms, where the master control unit is in communication connection with the mechanical arms, and the several mechanical arms are in an identical structure or different structures. The operation system for bone tissue has the beneficial effects as follows: the number of mechanical arms may be increased according to the requirements from implantation, osteotomy or other functional operations, and various use requirements may be met.

FIG. 18 is a structural schematic diagram of another osteotomy device in an embodiment of the present disclosure.

The osteotomy device shown in FIG. 18 differs from the osteotomy device shown in FIG. 15 in that the osteotomy device shown in FIG. 18 further includes a rotary joint portion consisting of a first rod portion 510 and a second rod portion 530 which are movably connected, and a first ball joint portion 540 and a second ball joint portion 550 which are arranged at two ends of the rotary joint portion, and the first ball joint portion 540 is movably connected to the observation portion 240 to adjust the orientation of the observation portion 240.

In some embodiments of the present disclosure, a joint location of the rotary joint is movably connected to a locking portion for adjusting a relative location between the rotary joint portion and the observation portion 240.

Specifically, with reference to FIG. 18, the locking portion 520 is provided at the joint of the first rod portion 510 and the second rod portion 530.

Operation steps of the osteotomy device in the embodiment of the present disclosure are described below with reference to FIGS. 3, 15 and 18.

After the actuator 210 is introduced into the vicinity of the vertebral pedicle 21 to be broken, the locking portion 520 is not locked, such that the observation portion 240 and the actuator 210 may move flexibly to find a location where osteotomy is required.

Then, locations, relative to the movable support (not shown in the figure), of the observation portion 240 and the actuator 210 are fixed by the locking portion 520, and finally, the osteotomy is performed by the actuator 210 with assistance of the observation portion 240.

Although the embodiments of the present disclosure have been described in detail above, it is apparent to those skilled in the art that various modifications and changes can be made to these embodiments. However, it should be understood that such modifications and changes are within the scope and spirit of the present disclosure described in the claims. Moreover, the present disclosure described herein can have other embodiments and can be performed or implemented in various ways.

What is claimed is:

1. An operation method for bone tissue, comprising:
S0: providing a first implantation device and a second implantation device, wherein the first implantation device comprises a hollowed structure, an adapter structure is arranged in the hollowed structure, and at least a portion of an outer side wall of a bottom of the second implantation device is provided with a corresponding adapter structure to be fixedly connected to the adapter structure;
S1: proceeding to a location step, wherein the first implantation device penetrates a first vertebral segment, so as to cause the hollowed structure to face an end face of a second vertebral segment and make surface contact with the second vertebral segment; and
S2: proceeding to a fixation step, wherein the second implantation device penetrates from the bottom thereof the hollowed structure to be fixedly connected to the second vertebral segment, and the second implantation device is fixedly connected to the hollowed structure by means of fixed connection between the corresponding adapter structure and the adapter structure.

2. The operation method for bone tissue according to claim 1, further comprising a vertebral segment spacing step after the location step in S1 and before the fixation step in S2, wherein the vertebral segment spacing step comprises:
adjusting a moving distance of the first implantation device towards the second vertebral segment, causing the hollowed structure to move towards the second vertebral segment, and spacing the first vertebral segment and the second vertebral segment by a vertebral segment distance.

3. The operation method for bone tissue according to claim 2, wherein during movement of the hollowed structure towards the second vertebral segment, the hollowed structure is controlled to face the end face of the second vertebral segment and keep surface contact with the second vertebral segment.

4. The operation method for bone tissue according to claim 1, wherein before the location step in S1 is performed, an osteotomy device is used to act on target bone tissue to form a space.

5. The operation method for bone tissue according to claim 4, wherein in the location step in S1, the step of causing the first implantation device to penetrate a first vertebral segment comprises:
causing the first implantation device to move from a location, near a transverse process, of a lamina in a direction towards the space until the first implantation device penetrates the first vertebral segment.

6. The operation method for bone tissue according to claim 1, wherein in the location step in S1, the hollowed structure is caused to face the end face of the second vertebral segment and make planar contact or spherical contact with the second vertebral segment.

7. The operation method for bone tissue according to claim 2, wherein before the location step in S1 is performed, the method further comprises a preoperative path planning step, the preoperative path planning step comprising:
providing three-dimensional model data and vertebral segment distance requirement data of target bone tissue;
constructing section data according to the three-dimensional model data;
forming second vertebral segment section contact data according to the vertebral segment distance requirement data and the section data; and
forming first vertebral segment implantation path data according to the second vertebral segment section contact data.

8. An operation system for bone tissue, comprising a first implantation device and a second implantation device, wherein the first implantation device comprises a hollowed structure, an adapter structure is arranged in the hollowed structure, and at least a portion of an outer side wall of a bottom of the second implantation device is provided with a corresponding adapter structure to be fixedly connected to the adapter structure.

9. The operation system for bone tissue according to claim 8, further comprising a master control unit and an implantation mechanical arm, the master control unit being in communication with the implantation mechanical arm; wherein
the implantation mechanical arm is provided with a clamping structure to adapt to the first implantation device and the second implantation device; and
the master control unit controls the implantation mechanical arm to clamp the first implantation device to penetrate a first vertebral segment and to be fixedly connected to same, so as to form a spacing distance between the first vertebral segment and a second vertebral segment, and further the master control unit controls the implantation mechanical arm to clamp the second implantation device to penetrate the first implantation device and to be fixedly connected to same and the second vertebral segment.

10. The operation system for bone tissue according to claim 9, further comprising an auxiliary image device in communication with the master control unit, wherein the master control unit stores spacing distance range reference data:
the auxiliary image device acquires real-time location information of the first vertebral segment and the second vertebral segment and feeds same back to the master control unit; and
the master control unit determines whether the spacing distance conforms to the spacing distance range reference data according to the real-time location information.

11. The operation system for bone tissue according to claim 9, wherein the first implantation device comprises a nut structure provided with internal threads and external nut threads, a bottom of the nut structure comprises a nut contact surface, the second implantation device comprises a screw structure provided with external threads, and the external threads adapt to the internal threads; and
the master control unit controls, by means of the implantation mechanical arm, the nut structure to penetrate the first vertebral segment, to pass through a space to make surface contact with the second vertebral segment to form the spacing distance and to be fixedly connected to the first vertebral segment, and further the master control unit controls, by means of the implantation mechanical arm, the screw structure to be placed into the second vertebral segment by means of the nut structure, and finally to be fixedly connected to the second vertebral segment by means of adaptation of the internal threads to the external threads.

12. The operation system for bone tissue according to claim 9, wherein the clamping structure of the implantation mechanical arm comprises a casing structure, a stretchable structure accommodated in the casing structure and a lock structure in movable contact with the stretchable structure, the casing structure being detachably and fixedly connected to the first implantation device, and the stretchable structure stretching out to push out the lock structure to abut against an inner wall of the first implantation device.

13. The operation system for bone tissue according to claim 12, wherein a top of the first implantation device is provided with an accommodation structure, and at least a portion of the lock structure pushed out as the stretchable structure stretches out is accommodated in the accommodation structure.

14. The operation system for bone tissue according to claim 13, wherein the clamping structure of the implantation mechanical arm further comprises an electromagnetic control portion electrically connected to the stretchable structure, and the electromagnetic control portion controls the lock structure to enter or exit the accommodation structure by controlling the stretchable structure to move towards or away from the first implantation device.

15. The operation system for bone tissue according to claim 8, further comprising an osteotomy device for acting on target bone tissue to form a space.

16. The operation system for bone tissue according to claim 15, further comprising a master control unit and an osteotomy mechanical arm clamping the osteotomy device, wherein the master control unit is in communication connection with the osteotomy mechanical arm, and the osteotomy device comprises an actuator clamped at a free end of the osteotomy mechanical arm, and the master control unit controls the osteotomy mechanical arm to cause the actuator to act on the target bone tissue to form the space.

17. The operation system for bone tissue according to claim 16, wherein the osteotomy mechanical arm further comprises a buffer mechanism partially sleeving the actuator, the buffer mechanism comprises a closed cavity and a drive portion penetrating the closed cavity, the drive portion divides the closed cavity into a first cavity and a second cavity, the actuator is sleeved with the drive portion, the first cavity is close to an actuation end of the actuator, and the second cavity is far away from the actuation end of the actuator.

18. The operation system for bone tissue according to claim 17, wherein the buffer mechanism further comprises a pressure sensing portion arranged on the actuator to acquire change of a pressure by the actuator acting on the target bone tissue.

19. The operation system for bone tissue according to claim 18, wherein the buffer mechanism further comprises a duct portion which is arranged in the closed cavity and is in communication with the first cavity and the second cavity separately, and the duct portion is provided with an opening and closing control portion which is in communication connection with the pressure sensing portion; and
when the pressure sensing portion detects that the pressure by the actuator acting on the target bone tissue is reduced to a minimum value within a first period as for the change of the pressure by the actuator acting on the target bone tissue, the opening and closing control portion closes communication of the duct portion with the first cavity and the second cavity, such that the drive portion produces a reverse thrust on the actuator in a direction opposite an operation direction to weaken the action of the actuator on the target bone tissue.

20. The operation system for bone tissue according to claim 8, further comprising a master control unit and several mechanical arms, wherein the master control unit is in communication connection with the mechanical arms, and the several mechanical arms are in an identical structure or different structures.
